(19) United States
(12) Patent Application Publication
(10) Pub. No.: US 2003/0118578 A1
(43) Pub. Date: Jun. 26, 2003

(54) Methods for treating ischemic reperfusion injury using IkappaB kinase-beta inhibitors

(76) Inventors: Anthony Rosenzweig, Newton, MA (US); Youngkeun Ahn, Kwangju (KR)

Correspondence Address: CLARK & ELBING LLP, 101 FEDERAL STREET, BOSTON, MA, 02110, US

(21) Appl. No.: 10/289,754

(22) Filed: Nov. 7, 2002

Related U.S. Application Data
(60) Provisional application No. 60/332,302, filed on Nov. 9, 2001.

(51) Int. Cl. A61K 48/00; A61K 38/53

(52) U.S. Cl. 424/94.5; 514/44

(57) ABSTRACT

The present invention relates to methods and compositions for reducing or preventing ischemia-reperfusion injury. Methods for identifying candidate compounds for such treatment are also described.

### CROSS REFERENCE TO RELATED APPLICATIONS

This application claims benefit of the filing date of the copending U.S. Provisional Application No. 60/332,302 (filed Nov. 9, 2001), hereby incorporated by reference.

### FIELD OF THE INVENTION

The field of the invention is the treatment of ischemic-reperfusion injury (IRI). In particular, the invention relates to methods for preventing or reducing IRI following ischemic episodes associated with, for example, myocardial infarction and organ transplantation. Further, methods are provided for identifying candidate compounds useful for treating or preventing IRI.

### BACKGROUND OF THE INVENTION

Cardiac infarction causes heart muscle death from ischemia (the deprivation of blood flow and oxygen). Paradoxically, restoring blood flow (reperfusion) may induce a complex series of events leading to both reversible and irreversible heart muscle damage, beyond any damage that may have occurred during the ischemic period. Most commonly, ischemia-reperfusion injury (IRI) occurs following a myocardial infarction (heart attack); however, IRI can occur following a cardiopulmonary bypass, during open-heart surgery, or in patients with unstable angina.

Ischemic episodes are not unique to cardiac tissue. The blood supply to many other tissues (e.g. liver, kidney, lungs, pancreas) is commonly interrupted by surgical procedures or disease states. Accordingly, there is a need to develop medications and treatment regimes that reduce or eliminate the effects of IRI. Such procedures will be particularly useful in treating patients suffering myocardial infarction, or interruptions in blood flow from surgery. Additionally, IRI prevention therapy can also be administered to patients undergoing organ transplantation or limb reattachment.

### SUMMARY OF THE INVENTION

In general, the present invention features a method for treating or preventing ischemic reperfusion injury to an organ in a mammal, by administering an IKK-β inhibitor to that organ. The IKK-β inhibitor is administered in an amount sufficient to reduce or prevent the ischemic reperfusion injury.

In preferred embodiments, the mammal is administered either a dominant negative IKK-β protein, or a nucleic acid capable of expressing a dominant negative IKK-β protein. Preferably, the mammal is a human, and the organ is a heart, liver, pancreas, or kidney.

The ischemic reperfusion injury treated or prevented by this method may be acute; for example, the ischemic reperfusion injury may result from a myocardial infarct. Alternatively, it may be iatrogenically-induced; for example, the ischemic reperfusion injury may result from cardiac surgery, coronary artery bypass surgery, valve replacement surgery, or percutaneous transluminal coronary intervention, including angioplasty or stenting. The iatrogenically-induced ischemic reperfusion injury may also result from organ transplantation.

In another aspect, the invention provides a method for identifying a candidate compound for reducing or preventing ischemic reperfusion injury. The method involves the steps of: (a) contacting an IKK-β expressing cell with a candidate compound; and (b) measuring IKK-β gene expression or IKK-β protein activity. A candidate compound that reduces the expression or activity of IKK-β, relative to a cell not contacted with the candidate compound, is identified as useful for reducing or preventing ischemic reperfusion injury.

In preferred embodiments, the IKK-β gene is an IKK-β fusion gene. In other embodiments, step (b) involves the measurement of IKK-β mRNA or protein.

In a related aspect, the invention provides another method for identifying a candidate compound for reducing or preventing ischemic reperfusion injury. This method involves the steps of: (a) contacting IKK-β protein with a candidate compound; and (b) determining whether the candidate compound binds the IKK-β protein and inhibits IKK-β kinase activity. Candidate compounds that bind and inhibit IKK-β kinase activity are identified as useful for reducing or preventing ischemic reperfusion injury.

In preferred embodiments, the method also tests the ability of the candidate compound to reduce expression of the IKK-β gene in a cell, for example, a mammalian cell such as a rodent or human cell. Most preferably, the IKK-β is human IKK-β.

The invention also provides a kit containing (a) a vector expressing a nucleic acid encoding a dominant negative IKK-β protein; and (b) instructions for delivery of the vector to an organ under conditions suitable for reducing or preventing ischemic reperfusion injury.

Accordingly, the invention also features a vector containing a polynucleotide that encodes a dominant negative IKK-β protein operably linked to a promoter. Preferably, the promoter is a target organ-specific promoter.

In preferred embodiments, the target organ is a heart, liver, kidney, or pancreas. Most preferably, the target organ is a human organ. Suitable heart-specific promoters for use with the vectors and kits of this invention include, for example, the myosin heavy chain promoter and the MCL_{2V} promoter.

As used herein, by “reducing or preventing ischemic-reperfusion injury” is meant ameliorating such injury before or after it has occurred. As compared with an equivalent untreated control, such reduction or degree of prevention is at least 5%, 10%, 20%, 40%, 50%, 60%, 80%, 90%, 95%, or 100% as measured by any standard technique.

By an “IKK-β inhibitor” is meant any compound that reduces the expression of an IKK-β gene or activity of an IKK-β protein. Preferably, such expression or activity is reduced by at least 2-fold, 3-fold, 5-fold, 10-fold, 20-fold, 50-fold, 100-fold, or even 1000-fold or greater.

By “dominant negative IKK-β” or “dnIKK-β” is meant any polypeptide with at least 50%, 70%, 80%, 90%, 95%, or even 99% sequence identity to human IKK-β, that maintains binding affinity toward wildtype IKK-β but dimerization results in a kinase-inactive product. The dnIKK-β used in the following experiments, has a K44A mutation; however, a skilled artisan will recognize that any insertion, deletion, or other mutation that imparts these properties, will function in an equivalent manner in the methods and compositions of this invention.

By “IKK-β fusion gene” is meant an IKK-β promoter and/or all or part of an IKK-β coding region operably linked to a second, heterologous nucleic acid sequence. In preferred embodiments, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and β-galactosidase.

By “acute” is meant a condition having a short course (for example, less than weeks or months), often sudden onset, and resulting from a disease process.

By “iatrogenically-induced” is meant a condition that is of longer duration than acute, and is planned, or is a consequence of a medical treatment (for example, a surgical technique).

By “reduces expression of an IKK-β gene or activity of an IKK-β protein” is meant to decrease expression or activity of IKK-β relative to control conditions. This reduction may be, for example, a decrease of least 2-fold, 3-fold, 5-fold, 10-fold, 100-fold, or even 1000-fold or greater, relative to control conditions.

By a “candidate compound” is meant a chemical, be it naturally-occurring or artificially-derived, that is surveyed for its ability to reduce IKK-β expression or activity by any standard assay method. Candidate compounds may include, for example, peptides, polypeptides, synthetic organic molecules, naturally-occurring organic molecules, nucleic acid molecules, and components thereof.

The present invention provides significant advantages over standard therapies for treatment or prevention of IRI. Currently, IRI therapy is focused on reducing the effects of inflammation and oxygen radical toxicity. Inhibition of IKK-β according to the present invention, prevents IRI-induced NF-κB activation, thereby reducing the pro-inflammatory signals and the overall amount of apoptosis of cardiomyocytes, resulting directly in a reduction of infarct size. In addition, the candidate compound screening methods provided by this invention allow for the identification of novel therapeutics that also act to modify the injury process, rather than merely mitigating the symptoms.

### BRIEF DESCRIPTION OF THE DRAWINGS

FIGS. 1A and 1B are Western blots showing that dnIKK-β blocks NF-κB activation in cardiomyocytes in vitro. (a) Immunoblotting for total IκB-α and phospho-IκB-α in cardiomyocytes. IκB-α degradation and phosphorylation of IκB-α after rat TNF-α stimulation (50 ng/mL, 10 min) were inhibited in Ad.dnIKK-β treated cells as compared with Ad.EGFP.β-gal infected cardiomyocytes. Data shown are representative of three independent experiments. (b) Immunoreactive nuclear p65-NF-κB in cardiomyocytes. Nuclear p65 nuclear translocation increased after rat TNF-α stimulation (50 ng/mL, 30 min) in Ad.EGFP.β-gal infected cells. This increase was inhibited in Ad.dnIKK-β transduced cardiomyocytes in a dose-dependent manner. Data shown are representative of three independent experiments.

FIG. 2 is a graph of amplification plots for VCAM-1 following quantitative RT-PCR in cells infected with Ad.dnIKK-β or control virus, and stimulated with TNF-α (50 ng/ml). After TNF-α treatment, there was a leftward shift of the amplification curve indicating significant induction of VCAM-1 mRNA in Ad.EGFP.β-gal infected CM (upper panel). This leftward shift was substantially inhibited in cells expressing dnIKK-β (lower panel). Similar plots were obtained for ICAM-1. In cumulative data from three independent experiments, VCAM-1 and ICAM-1 induction was significantly inhibited by dnIKK-β expression (p<0.05, for both), by 90±11% and 80±13%, respectively.

FIGS. 3A and 3B are photomicrographs of Western blots demonstrating that dnIKK-β inhibits NF-κB activation in vivo. (a) Immunoblotting for IκB-α. IαB-α degradation was evident after 30 min of ischemia followed by 30 min of reperfusion in Ad.EGFP.β-gal infected hearts compared with non-ischemic hearts. In contrast, Ad.dnIKK-β treatment reduced IκB-α degradation after 30 min ischemia and 30 min reperfusion. (b) Immunoblotting for nuclear p65 in myocardium Nuclear p65 increased after IR (30 min ischemia, 24 hr reperfusion) in Ad.EGFP.β-gal or buffer treated myocardium. This increase was substantially inhibited in Ad.dnIKK-β treated myocardium despite IR. All blots are representative of three independent experiments.

FIG. 3C shows confocal microscopy on immunocytochemical preparations of Ad.dnIKK-β treated myocardium after IR. Confocal microscopy for GFP which is co-expressed by Ad.dnIKK-β (left panel) or immunoreactive p65 (right panel) after IR (30 min ischemia, 24 hr reperfusion) revealed that p65 remained predominantly in the cytoplasm in Ad.dnIKK-β transduced (GFP-expressing) cells but moved to the nucleus in cells not expressing the transgene (seen in bottom half of right panel). Data shown are representative of three independent experiments.

FIG. 4 is a bar graph showing that MCP-1 induction is blocked by dnIKK-β after IR in vivo. Tissue MCP-1 was measured by ELISA from normal myocardium (NL) or hearts infected with Ad. EGFP.β-gal or Ad.dnIKK-β and subjected to IR (30 min ischemia, 24 hr reperfusion). MCP-1 increased significantly in the ischemic regions (I) of hearts treated with Ad.EGFP.β-gal, compared to non-ischemia regions (N) (p<0.05). In contrast, MCP-1 concentration was significantly lower in the ischemic regions from Ad.dnIKK-β treated hearts compared with the ischemic regions from Ad.EGFP.β-gal infected hearts (p<0.05), and not significantly different from non-ischemic or normal myocardium (p=NS). Data shown are cumulative with four animals in each group.

FIG. 5A is a bar graph showing myocardial MPO activity. As an index of neutrophil infiltration, MPO activity was measured in ischemic and non-ischemic regions after IR (30 min ischemia, 24 hr reperfusion). MPO increased significantly in the ischemic regions (I) from Ad.EGFP.β-gal treated animals, compared to non-ischemic (N) or normal (NL) myocardium (*p<0.01). In Ad.dnIKK-β treated rats, this increase was significantly reduced compared with Ad.EGFP.β-gal treated animals (**p<0.05) but remained above the level seen in non-ischemic regions or control myocardium (**p<0.05). Data shown is cumulative with four animals in each group.

FIG. 5B is a photomicrograph of H&E staining of tissue from Ad.EGFP.β-gal and Ad.dnIKK-β treated myocardium after IR. Many neutrophils are evident infiltrating ischemic tissue from Ad.EGFP.β-gal treated myocardium (left panel), while fewer neutrophils are seen in the ischemic tissue from Ad.dnIKK-β treated animals (right panel). Data shown are representative of four independent experiments

FIG. 6 is a bar graph and photomicrographs demonstrating that Ad.dnIKK-β reduces infarction after IR. Representative micrograph (right panel) revealing fluorescent microsphere distribution (top) and TTC staining (bottom) from rats subjected to IR after gene transfer with Ad.EGFP.β-gal (left) or Ad.dnIKK-β (right). Bar graph shows cumulative data for AAR and % MI from Ad.EGFP.β-gal and Ad.dnIKK-β treated animals (n=8 in each group) after IR. There was no significant difference in the ischemia area (AAR) between the groups. In contrast, infarction (% MI) was reduced by 78% in Ad.dnIKK-β treated animals (p<0.001).

FIG. 7 is an agarose gel showing reductions of DNA laddering in dnIKK-β expressing myocardium. DNA isolated from ischemic (I) and non-ischemic (N) regions of hearts after IR (30 min ischemia, 24 hr reperfusion) was subjected to gel electrophoresis. No DNA laddering was evident in the non-ischemic regions of any of the groups. A significant increase in DNA laddering was evident in ischemic regions of animals treated with buffer alone or Ad.EGFP.β-gal. Laddering was attenuated in the ischemic region from animals treated with Ad.dnIKK-β. Data shown are representative of four independent experiments.

### DETAILED DESCRIPTION

The nuclear factor kappa B (NF-κB) family of transcription factors is activated by diverse stimuli, including oxidative stress and inflammatory cytokines, and drives expression of many genes involved in inflammation and cell survival. NF-κB generally exists as a dimer in the cytosol bound to one of three inhibitory, IκB, subunits. A major mechanism of NF-κB activation is serine phosphorylation and degradation of IκB , followed rapidly by translocation of NF-κB to the nucleus where it activates transcription of specific promoter targets. In addition, some members of the NF-κB family, such as p65, can be regulated through direct phosphorylation of the transactivation domain (TAD), further enhancing gene transcription. Two known kinases, IKK-α and IKK-β, can each phosphorylate IκB . Mice lacking IKK-β die as embryos but their embryonic fibroblasts have defective NF-κB activation in response to cytokine stimulation. IKK-α appears important for activation of one of the NF-κB family members (NF-κB2) and a subset of NF-κB dependent genes, particularly in B cells. IKK-α is also important in skin development but this function is independent of its kinase activity. Recent data also suggests reversible acetylation of p65 also modulates its association with IκB and transcriptional activity. Thus, multiple mechanisms of NF-κB regulation seem to exist.

IRI is a common consequence of myocardial infarction, organ transplantation, limb reattachment, and iatrogenic or idiopathic disruptions of blood flow. To test the role of IKK-β in cardiac IRI, we used adenoviral gene transfer of a highly specific and effective dominant negative IKK-β mutant (dnIKK-β) and examined the effects on NF-κB activation, inflammation, apoptosis, and cardiac injury. Our results demonstrate that IKK-β inhibition is an effective strategy to attenuate IRI.

Recombinant Adenoviral Vectors

Two recombinant type 5 adenoviruses (Ad.EGFP.β-gal and Ad.dnIKK-β) were used in these studies. Ad.EGFP.β-gal has been described in detail by Matsui et al. (Circulation (2001) 104:330-335). Ad.dnIKK-β was constructed by subcloning the cDNA for the kinase-inactive mutant (K44A) of IKK-β with a carboxy-terminal Flag epitope into the shuttle plasmid, pAdTrack-CMV, which also encodes a separate expression cassette for CMV-driven EGFP expression. Full length adenoviral DNA clones, incorporating this shuttle vector, were obtained through homologous recombination with pAdEasy-1 in E. coli (BJ5183) and prepared as high titer stocks, as described by He et al. (Proc. Natl. Acad. Sci. USA (1998) 95:2509-14). Adenoviral vectors were amplified in 293 cells, particle count estimated from OD₂₆₀ and titer determined by plaque assay. Stock titers were >10⁹ pfu/ml for each vector with a particle/pfu ratio of about 20-50. Vector doses are expressed as multiplicity of infection (MOI), defined as plaque-forming units per cell. Wild-type adenovirus contamination was excluded by the absence of PCR-detectable E1 sequences.

Neonatal Cardiomyocytes

Cardiomyocytes (CM) were prepared from 1-2 day-old rats as described by Matsui et al. (Circulation (1999) 100:2373-9). Neonatal CM grown in 60 mm dishes were infected with Ad.EGFP.β-gal (MOI 20), or Ad.dnIKK-β (MOI 20), for 24 hours prior to activation with recombinant rat TNF-α (50 ng/mL). Nuclear and cytoplasmic extracts were prepared.

Animal Studies

Male Sprague-Dawley rats weighing 250-300 g were subjected to in vivo gene transfer with Ad.EGFP.β-gal, or Ad.dnIKK-β, 48 hours prior to IR (30 minutes left anterior descending artery (LAD) ligation, 24 hours reperfusion, unless otherwise indicated). To evaluate early changes in IκB , some animals were evaluated after 30 minutes of ischemia and only 30 minutes of reperfusion. Rats were sacrificed 24 hours after ischemia and the infarct area (% MI) as a proportion of the area-at-risk (AAR) was determined as described by Matsui et al. (Circulation (2001) 104:330-335).

DNA Laddering

Fresh tissues were microdissected under UV light into ischemic and non-ischemic regions. All tissue from each region was lysed (100 mM Tris (pH 8.5), 5 mM EDTA, 0.2% SDS, 200 mM NaCl, 100 μg/mL proteinase K) at 37° C. for 18 to 20 hours. DNA was prepared, labeled with [α-³²P]dCTP, subjected to electrophoresis and autoradiography.

Immunohistochemistry and H&E

Hearts were fixed in 4% paraformaldehyde. Five micron sections were treated with 0.1% SDS and incubated with primary antibody to NF-κB p65 for 90 minutes at 37° C. Sections were rinsed in PBS and incubated with anti-mouse IgG conjugated to tetramethyl rhodamine (60 minutes, 37° C.). Confocal images were obtained using a laser confocal system. Hematoxylin and eosin (H&E) staining was performed for histomorphologic evaluation of neutrophil infiltration.

Western Blotting

Proteins were separated by SDS-PAGE performed under reducing conditions on 7.5%, 10%, and 12% separation gels with a 4% stacking gel. Proteins were transferred to nitrocellulose membranes by semi-dry blotting. Membranes were incubated with primary antibodies to IκB-α, phosph-IκB-α (Ser 32), NF-κB p65, or IKK-β overnight at 4° C. After washing, membranes were incubated with horseradish peroxidase-conjugated secondary antibody and immunoreactive bands detected by chemiluminescence.

Tissue Myeloperoxidase Activity (MPO)

Myocardial MPO activity was determined as an index of neutrophil infiltration. Frozen normal, ischemic, and non-ischemic heart samples (20 mg) were homogenized in 50 mmol/L potassium phosphate buffer (PPB). After centrifugation (12,500×g, 20 minutes, 4° C.), pellets were resuspended in PPB containing 0.5% hexadecyltrimethyl ammonium bromide (HTAB) (Sigma). Samples were sonicated on ice, freeze-thawed, and centrifuged (12,500×g, 20 minutes, 4° C.). Supernatants were collected and incubated with reaction buffer (0.167 mg/mL of o-dianisidine dihydrochloride, 0.0005% H₂O₂, 50 mM PPB). Absorbance was measured spectrophotometrically at a wavelength of 470 nm. MPO activity was expressed as OD_{(sample-blank)}/mg protein/minute.

MCP-1 ELISA

Myocardial homogenates were suspended in PBS solution containing protease inhibitors (PMSF 1 mM, leupeptin 1 μg/mL, aprotinin 1 μg/mL) and 1% Triton-X100. After incubation (1 hour, 4° C.), extracts were centrifuged (20,000×g, 20 minutes, 4° C.) to remove cellular debris. Expression of rat MCP-1 was quantified by ELISA.

Quantitative RT-PCR

Neonatal CM were incubated with TNF-α (50 ng/mL, 3 hrs) and then harvested in Trizol reagent. Samples were centrifuged (12,000×g, 10 minutes, 4° C.), supernatants were removed and vortexed (20 seconds) with an equal volume of isopropanol. Total RNA was precipitated by centrifugation (12,000×g, 10 minutes, 4° C.) and purified. Expression of the VCAM-1, ICAM-1, and MCP-1 in samples was determined using quantitative RT-PCR analysis and sequence-specific primers. RNA (100 ng/reaction) was reverse transcribed and the cDNA subsequently amplified.

Adenoviral Gene Transfer in Vivo

Direct injection of adenoviral vectors resulted in regional transgene expression in about 60% of the ischemic area. Expression of the appropriate size protein was detected by immunoblotting with IKK-β specific antibodies only in Ad.dnIKK-β injected myocardium.

dnIKK-β Blocks IκB-α Phosphorylation and NF-κB Activation in Cardiomyocytes

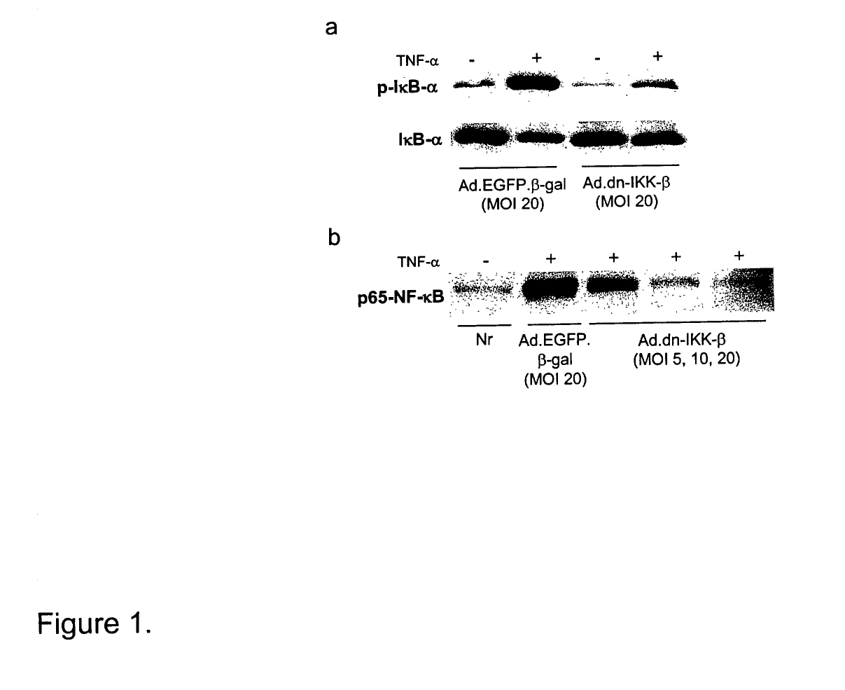

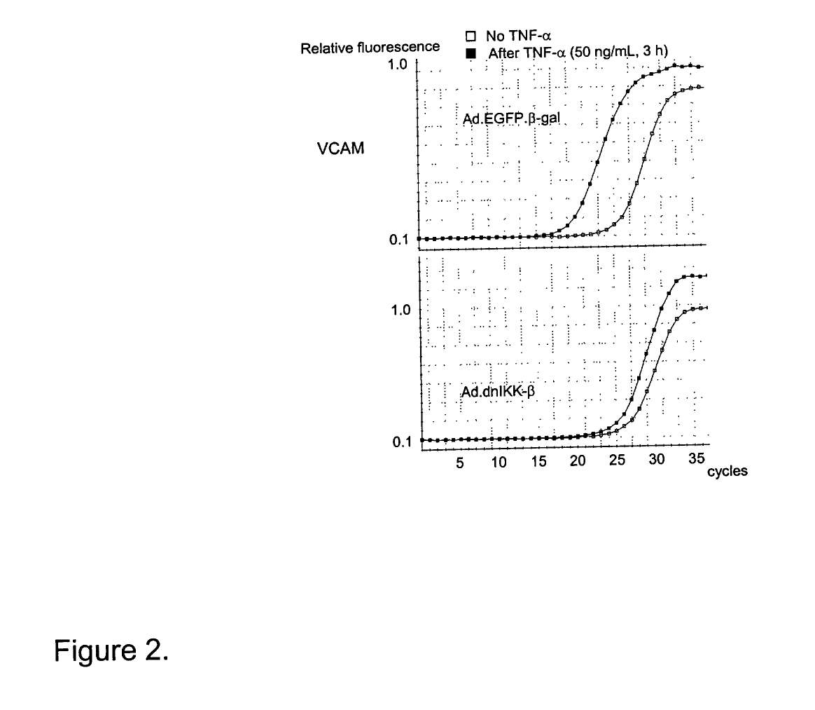

We examined whether dnIKK-β expression would block IκB phosphorylation and NF-κB activation in CM in vitro. Cultured CM that had been transduced with Ad.EGFP.β-gal or Ad.dnIKK-β for 24 hours were treated with recombinant rat TNF-α (50 ng/mL) for 10 to 30 minutes. At 10 minutes, TNF-α stimulation induced a significant increase in immunoreactive phospho-IκB-α and a decrease in total IκB-α in Ad.EGFP.β-gal infected cells (FIG. 1a). In contrast, IκB -α and phospho-IκB-α levels were only minimally affected by TNF-α treatment in Ad.dnIKK-β infected cells (FIG. 1a). By 30 minutes, rat TNF-α induced a significant increase in nuclear p65-NF-κB in Ad.EGFP.β-gal infected cells (FIG. 1b). This increase was significantly blocked by dnIKK-β expression in a dose-dependent manner (FIG. 1b). To determine whether transcription of NF-κB dependent genes was effectively blocked, we performed quantitative RT-PCR (QRT-PCR) on RNA (100 ng) from CM infected with Ad.dnIKK-β or Ad.EGFP.β-gal and stimulated with TNF-α (50 ng/mL, 3 hours). Amplified product was detected using SYBR1 fluorescence (FIG. 2). Importantly, post-PCR melt curve analysis confirmed a single peak of amplified product of the appropriate Tₘ and there was no amplification in the absence of template. After TNF-α treatment, there was a leftward shift of the amplification curves indicating a significant increase in mRNA levels for each of the examined genes in Ad.EGFP.β-gal infected CM. This leftward shift was substantially inhibited in cells expressing dnIKK-β (FIG. 2). Overall, dnIKK-β inhibited the induction of mRNA for VCAM-1 and ICAM-1 (p<0.05) by 90±11% and 80±13%, respectively. In two additional experiments, induction of MCP-1 was also inhibited in dnIKK-β-expressing CM by an average of 51±9%.

dnIKK-β Inhibits NF-κB Activation in Vivo

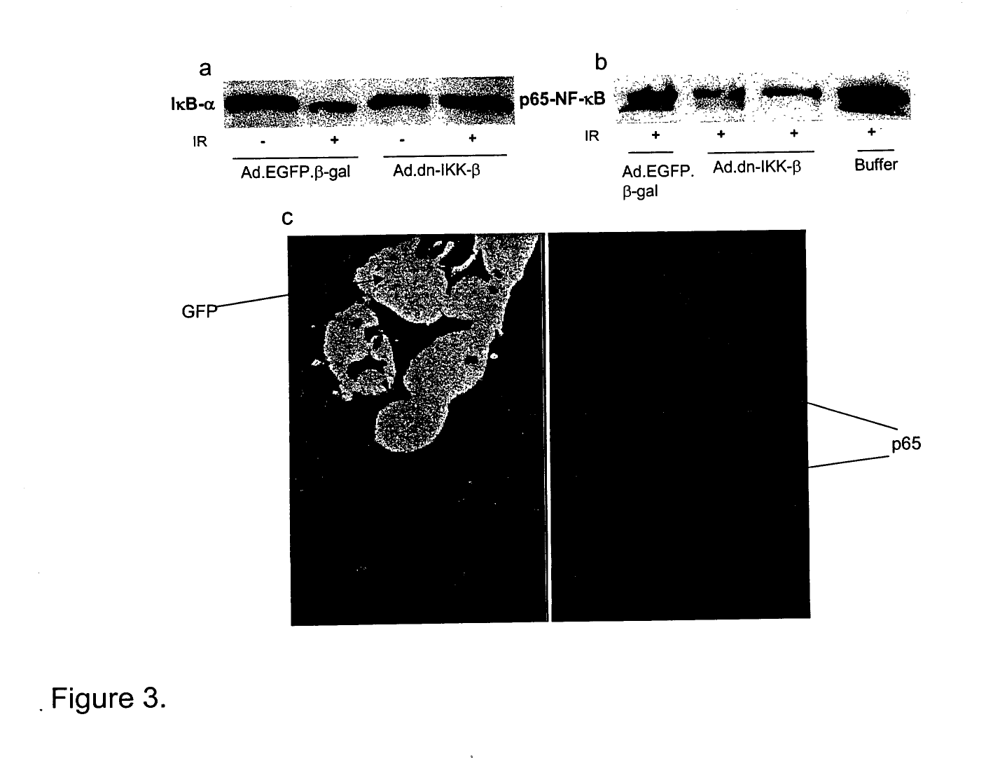

To investigate whether dnIKK-β expression could block NF-κB activation after IR in vivo, we examined myocardial nuclear and cytoplasmic proteins isolated from the ischemic areas of hearts subjected to 30 minutes of ischemia and 30 minutes of reperfusion for IκB-α, as well as 30 minutes of ischemia and 24 hours of reperfusion for nuclear p65-NF-κB. Immunoreactive IκB-α decreased in Ad.EGFP.β-gal treated hearts after IR. This decrease was blocked by dnIKK-β expression (FIG. 3a). Similarly, an increase in nuclear p65 was evident in Ad.EGFP.β-gal or buffer treated rats by immunoblotting after IR. In contrast, p65 nuclear translocation was blocked in Ad.dnIKK-β treated rats (FIG. 3b). In Ad.dnIKK-β injected myocardium, inhibition of p65 nuclear translocation was also evident on immunohistochemical examination. Confocal microscopy for GFP which is co-expressed by Ad.dnIKK-β (left panel) or immunoreactive p65 (right panel, arrows) revealed that p65 remained predominantly in the cytoplasm of Ad.dnIKKβ-transduced (GFP-expressing) cells but moved to the nucleus in cells not expressing the transgene (seen in bottom half of right panel) (FIG. 3c).

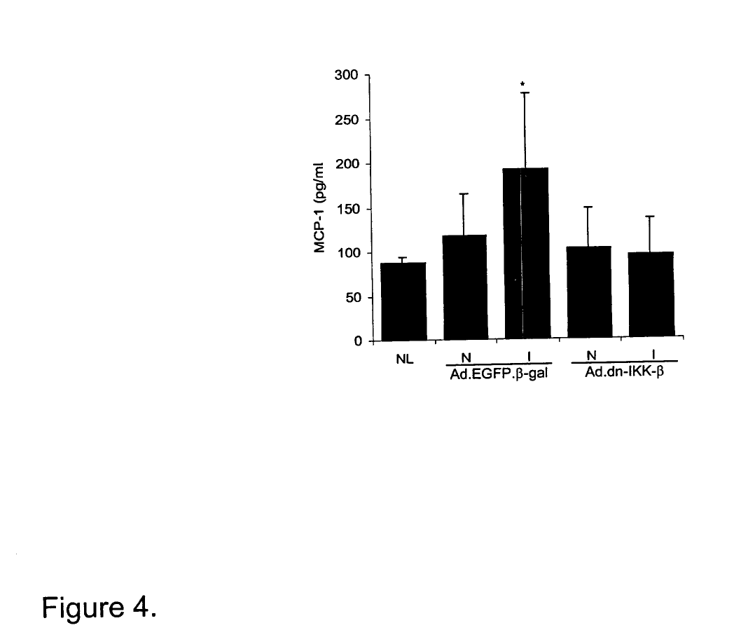

We also examined the effect of dnIKK-β expression on in vivo induction of the NF-κB-dependent inflammatory chemokine, MCP-1, since a quantitative ELISA assay specific for rat MCP-1 is commercially available. Tissue MCP-1 expression increased after IR in ischemic myocardium in Ad.EGFP.β-gal treated rats compared with normal hearts (87.3±7.3 vs. 192.6±85.1 pg/ml, p<0.05, n=4 in each group). This increase was completely blocked in Ad.dnIKK-β treated animals (95.2±41.8 vs. 192.6±85.1 pg/ml, p<0.05), reducing chemokine concentration to levels seen in non-ischemic or normal myocardium (FIG. 4).

Ad. dnIKK-β Reduces Neutrophil Infiltration After IR

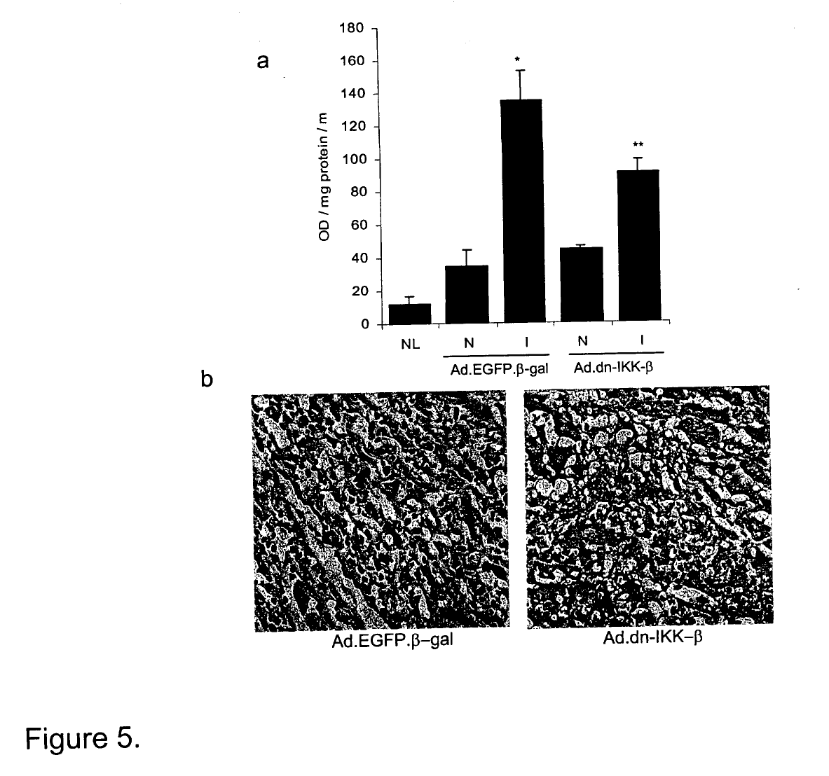

NF-κB activation of inflammatory pathways are thought to contribute to IR injury through recruitment of neutrophils which mediate, at least in part, myocardial injury. Leukocyte infiltration into damaged myocardium following IR was assessed by measurement of MPO activity, a specific marker for neutrophils. MPO activity was increased in ischemic regions following 30 minutes of ischemia and 24 hours of reperfusion in both Ad.EGFP.β-gal and Ad.dnIKK-β treated rats compared with normal hearts. However, MPO activity in the ischemic area was decreased by 33% in the Ad.dnIKK-β treated rats compared with Ad.EGFP.β-gal treated rats (90.7±7.8 vs. 134.9±17.9 OD/minute/mg protein, p<0.05, n=4 in each group) (FIG. 5a). These data were consistent with the results of histological evaluation of H&E-stained myocardium. Neutrophils were rarely seen in non-ischemic myocardium from either group (data not shown), but were readily detected in ischemic myocardium. Neutrophil infiltration was less prominent in Ad.dnIKK-β treated myocardium compared with Ad.EGFP.β-gal treated myocardium (FIG. 5b).

Infarct Size and Area at Risk

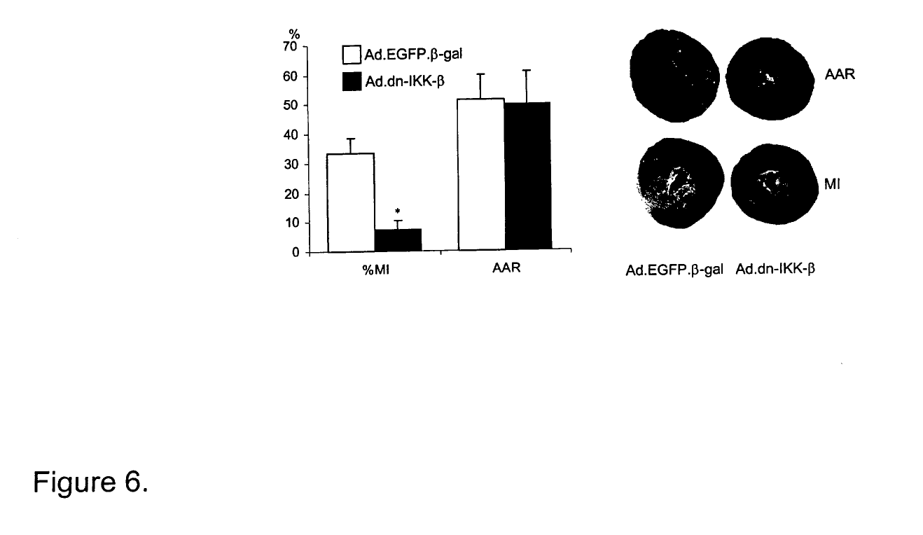

To determine the impact of the observed reduction in inflammation on clinically relevant endpoints, we examined cumulative ischemic and infarcted areas in hearts from animals treated with Ad.dnIKK-β or Ad.EGFP.β-gal. The ischemic area induced by LAD ligation (AAR) did not differ between the animals infected with the Ad.dnIKK-β or Ad.EGFP.β-gal. However, Ad.dnIKK-β treated rats demonstrated a dramatic 78% reduction in infarct size (% MI) compared with the Ad.EGFP.β-gal treated rats (7.3±2.1% vs. 33.3±5.4%, p<0.001, n=8 in each group) (FIG. 6).

Apoptosis

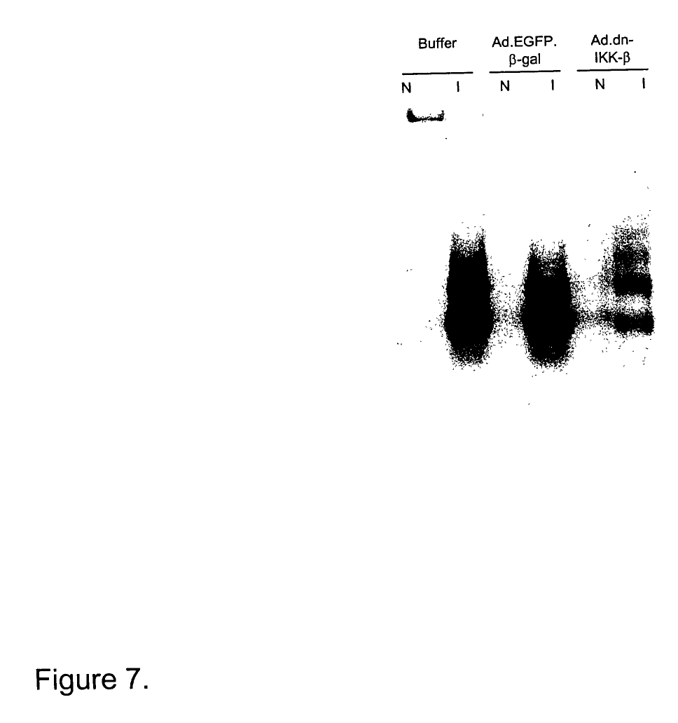

Since apoptosis can contribute to myocardial injury in IR, and CM require NF-κB-dependent survival factors in some settings, we examined the effect of dnIKK-β expression on DNA laddering, a biochemical hallmark of apoptosis. Left ventricular (LV) samples were divided into ischemic and non-ischemic areas, delineated by fluorescent microsphere distribution, DNA extracted, and subjected to agarose gel electrophoresis. DNA laddering was not observed in the non-ischemic myocardium of animals treated with buffer alone, Ad.EGFP.β-gal, or Ad.dnIKK-β (n=4 in each group). However, prominent DNA laddering was evident in ischemic myocardium from buffer and Ad.EGFP.β-gal treated rats. This laddering was markedly attenuated by dnIKK-β expression (FIG. 7).

Expression of dnIKK-β for Treating Ischemia-Reperfusion Injury

IRI can result from a variety of planned and unplanned ischemic episodes. Gene transfer of dominant negative IKK-β can be used prophylactically or therapeutically in numerous circumstances. These include the following examples.

(1) Acute coronary syndromes. Gene delivery at the time of presentation limits inflammation and injury over the ensuing hours to days, and reduces the adverse remodeling that occurs during the weeks and months following infarction.

(2) Coronary artery bypass surgery or valve replacement surgery. These procedures are associated with transient ischemia because of imperfect perfusion during pump perfusion or absent perfusion if hypothermic arrest is utilized.

(3) Percutaneous transluminal coronary interventions (PCI) including, for example, angioplasty and stenting.

(4) Transplantation of the heart or other organs (e.g. liver, kidney, pancreas). There is a significant ischemic time associated with organ harvest and transport, followed by reperfusion after vascular anastomoses are established. Expression of dnIKK-β will minimize tissue injury resulting from reperfusion injury, and promote organ donor viability.

In cases of iatrogenically-induced ischemia (e.g. cases 2-4), dnIKK-β gene transfer can be performed prior to the procedure.

Vectors for Cardiac Gene Transfer

Cardiac gene transfer requires suitable vector and delivery systems. The most common are plasmid (“naked”) DNA, adenoviral vectors, or adeno-associated viral vectors (AAV).

Plasmid DNA

Plasmid DNA is often referred to as “naked DNA” because of the absence of a more elaborate packaging system. The heart has the ability to take up and express genes directly injected as plasmids (Lin, et al., Circulation (1990) 82:2217-21; Kitsis, et al., Proc Natl Acad Sci USA. (1991) 88:4138-42; Gal, et al., Lab. Invest. (1993) 68:18-25). This has been demonstrated both in animal models and clinically (Takeshita, et al., Am. J. Pathol. (1995) 147:1649-60; Losordo, et al., Circulation (1998) 98:2800-4).

Adenoviruses

Recombinant adenoviral vectors offer several significant advantages for cardiac gene transfer. The viruses can be prepared at extremely high titer, infect non-replicating cells, and confer high-efficiency and high-level transduction of cardiomyocytes in vivo after direction injection or perfusion. Either direct injection or perfusion would be appropriate for delivery of dnIKK-β vectors in a clinical setting. For transient ischemia or IRI, transient expression is sufficient because it minimizes biosafety or toxicity concerns.

In animal models, adenoviral gene transfer to adult myocardium in vivo has generally been found to mediate high-level expression for approximately one week. The duration of transgene expression may be prolonged and ectopic expression reduced by using cardiac specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a Cre-Lox strategy (Engelhardt, et al., Proc. Natl. Acad. Sci. USA. (1994) 91:6196-200; Kochanek, et al. Proc. Natl. Acad. Sci. USA (1996) 93:5731-6). Ideally, dnIKK-β expression would be mediated by one of these later generation adenoviral vectors utilizing a cardiac-specific promoter.

Adeno-associated Viruses

Recombinant adeno-associated viruses (rAAV), derived from non-pathogenic parvoviruses, evoke almost no cellular immune response, and produce transgene expression lasting months in most systems. Accordingly, rAAVs are promising for sustained cardiac gene transfer. Incorporation of a cardiac-specific promoter is, again, beneficial.

Other vectors and techniques are known in the art. For example, see Wattanapitayakul and Bauer (Biomed. Pharmacother. (2000) 54: 487-504), and citations therein.

Delivery of Vectors to the Heart

A vector carrying dnIKK-β can be delivered to the heart (or other target organ) hours, days, or even weeks before an anticipated episode of IRI (e.g. transplantation). Several approaches have been utilized to successfully deliver transgenes to the heart in vivo. Some of the techniques used have been intracoronary catheter delivery (Barr, et al., Gene Ther. (1994) 1:51-58; Donahue, et al., Proc. Natl. Acad. Sci. USA (1997) 94:4664-8), direct injection of the vector into the myocardium (Rosengart, et al., Ann. Surg. (1999) 230:466-70; Circulation (1999) 100:468-74), intraventricular delivery with retroinfusion of the coronary veins (Boekstegers, et al., Gene Ther. (2000) 7:232-40), and injection of adenovirus into the pericardial sac (Fromes, et al., Gene Ther. (1999) 6:683-8). Other techniques deliver vector more globally through in vivo perfusion by injecting the vector into the aortic root, just above the aortic valve, while the aorta and pulmonary artery are transiently cross-clamped (Hajjar, et al., Proc. Natl. Acad. Sci. USA. (1998) 95:5251-6). This technique achieves homogeneous transduction of the myocardium, and has also been shown to produce transgene-specific physiological effects on ventricular function in vivo. In addition, vector can also be delivered at the time of bypass surgery utilizing the pump perfusion system to distribute the virus to the heart. Because pump time can last hours, instillation of dnIKK-β vector at the initiation of the run could provide time for transgene expression during the reperfusion phase most subject to IRI.

Other Organs

Similar principles apply to IRI in other organs, including for example, liver, lungs, kidney, and pancreas. Gene transfer is particularly applicable during organ transplantation because transfection could be done in vivo, in the donor, prior to organ harvest. Alternatively, the isolated organ can be after harvest. Ex vivo perfusion or direct injection has previously been used successfully for gene transfer in models of transplantation in combination with viral and other gene transfer vectors. Although conceptually similar, in these cases the vector is delivered either by direction injection into the non-cardiac organ or by delivery into the blood vessels supplying this organ (e.g., for the liver either the portal vein (Tada, et al. Liver Transpl. Surg. (1998) 4:78-88)) or the hepatic artery (Habib, et al. Hum. Gene Ther. (1999) 10:2019-34) could be used.

Identification of Candidate Compound for the Treatment or Prevention of IRI

A candidate compound that is beneficial in the treatment or prevention of IRI can also be identified using IKK-β as the drug target. For example, a candidate compound can be identified by its ability to affect the biological activity of IKK-β or the expression of the IKK-β gene. Compounds that are identified by the methods of the present invention, that reduce the biological activity or expression levels of IKK-β, represent candidate compounds or lead compounds for the treatment or prevention of IRI.

Expression of a reporter gene that is operably linked to an IKK-β promoter, or portion of the IKK-β coding sequence, can be used to identify such candidate compounds. A reporter gene may encode a reporter enzyme that has a detectable read-out, such as beta-lactamase, beta-galactosidase, or luciferase. Reporter enzymes can be detected using methods known in the art, such as the use of chromogenic or fluorogenic substrates for reporter enzymes as such substrates are known in the art. Such substrates are desirably membrane permeant. Chromogenic or fluorogenic readouts can be detected using, for example, optical methods such as absorbance or fluorescence. A reporter gene can be part of a reporter gene construct, such as a plasmid or viral vector, such as a retrovirus or adeno-associated virus. A reporter gene can also be extra-chromosomal or be integrated into the genome of a host cell. The expression of the reporter gene can be under the control of exogenous expression control sequences or expression control sequences within the genome of the host cell. Under the latter configuration, the reporter gene is desirably integrated into the genome of the host cell.

Any assay that measures the kinase activity of IKK-β can also be used to identify candidate compounds. Desirably, IκB -α is used as the substrate to measure IKK-β phosphorylation activity. As described above, phospho-IκB-α can be measured in CM cells exposed to a candidate compound. Alternatively, phosphorylation of IκB-α by IKK-β can be directly assessed in a cell-free assay. For example, purified, recombinant IκB-α can be phosphorylated by IKK-β, in vitro, in a standard [³²P]-dATP kinase assay. The reaction products can be detected by scintillation spectroscopy.

A candidate compound identified by the methods of the present invention can be from natural as well as synthetic sources. Those skilled in the field or drug discovery and development will understand that the precise source of test extracts or compounds is not critical to the methods of the invention. Examples of such extracts or compounds include, but are not limited to, plant-, fungal-, prokaryotic-, or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Screening methods according to the invention may be carried out in any cell, for example, a cell (such as a mammalian cell) into which a heterologous IKK-β gene or a IKK-β reporter gene has been introduced. Alternatively, these screens may be carried out in cells in which the IKK-β gene is overexpressed, or has increased activity. In these cells, compounds that reduce IKK-β activity can be identified. Desirable candidate compounds are identified as those which reduce phosphorylation of IκB , NF-κB activity, or reduce IKK-β expression or activity.

Administration of dnIKK-β or a Candidate Compound for Treatment or Prevention of IRI

The present invention further includes methods for treating or preventing IRI by administering a dnIKK-β polypeptide or other compound that inhibits IKK-β expression or activity. The administration of a dnIKK-β polypeptide that, regardless of its method of manufacture, inhibits biological activity of an endogenous pair member, can be utilized to reduce IKK-β biological activity in a patient suffering from IRI, following, for example, an ischemic attack or in preparation for ischemic reperfusion injury, such as is associated with an organ transplant. Alternatively, a compound that compensates for, or inhibits, IKK-β expression or activity can be similarly used.

Peptide agents of the invention, such as a dnIKK-β polypeptide, or a candidate compound can be administered to a subject, e.g., a human, directly or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. One exemplary pharmaceutically acceptable carrier is physiological saline. Other physiologically acceptable carriers and their formulations are known to one skilled in the art and described, for example, in Remington's Pharmaceutical Sciences, (19th edition), ed. A. Gennaro, 1995, Mack Publishing Company, Easton, Pa.

Pharmaceutical formulations of a therapeutically effective amount of a peptide agent or candidate compound of the invention, or pharmaceutically acceptable salt-thereof, can be administered orally, parenterally (e.g. intramuscular, intraperitoneal, intravenous, subcutaneous, or intracardiac injection), in admixture with a pharmaceutically acceptable carrier adapted for the route of administration.

Methods well known in the art for making formulations are found, for example, in Remington's Pharmaceutical Sciences (19th edition), ed. A. Gennaro, 1995, Mack Publishing Company, Easton, Pa. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polypeptides of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The amount of active ingredient in the compositions of the invention can be varied. One skilled in the art will appreciate that the exact individual dosages may be adjusted somewhat depending upon a variety of factors, including the polypeptide or compound being administered, the time of administration, the route of administration, the nature of the formulation, the rate of excretion, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Intracardiac injection would, presumably, require the lowest dosage. Variations in these dosage levels can be adjusted using standard empirical routines for optimization, which are well known in the art. In general, the precise therapeutically effective dosage will be determined by the attending physician in consideration of the above identified factors.

The polypeptide or candidate compound of the present invention can be prepared in any suitable manner. It can be isolated from naturally occurring sources, recombinantly produced, or produced synthetically, or produced by a combination of these methods. The synthesis of short peptides is well known in the art. See e.g. Stewart et al., Solid Phase Peptide Synthesis (Pierce Chemical Co., 2d ed., 1984). Additionally, polypeptides (e.g. dnIKK-β) may be post-translationally modified to promote cellular uptake, enhance biological activity, or improve the pharmacokinetic profile.

Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for reducing or preventing ischemic reperfusion injury to an organ in a mammal, said method comprising administering to said organ an IKK-β inhibitor in an amount sufficient to reduce or prevent said ischemic reperfusion injury.

2. The method of claim 1, wherein said IKK-β inhibitor is a dominant negative IKK-β protein.

3. The method of claim 1, wherein said IKK-β inhibitor is a nucleic acid expressing a dominant negative IKK-β protein.

4. The method of claim 1, wherein said mammal is a human.

5. The method of claim 1, wherein said organ is a heart.

6. The method of claim 1, wherein said ischemic reperfusion injury is acute.

7. The method of claim 6, wherein said acute ischemic reperfusion injury results from a myocardial infarct.

8. The method of claim 1, wherein said ischemic reperfusion injury is iatrogenically-induced.

9. The method of claim 8, wherein said iatrogenically-induced ischemic reperfusion injury results from cardiac surgery.

10. The method of claim 9, wherein said cardiac surgery is coronary artery bypass surgery or valve replacement surgery.

11. The method of claim 8, wherein said iatrogenically-induced ischemic reperfusion injury results from a percutaneous transluminal coronary intervention.

12. The method of claim 11, wherein said percutaneous transluminal coronary intervention is angioplasty or stenting.

13. The method of claim 8, wherein said iatrogenically-induced ischemic reperfusion injury results from organ transplantation.

14. The method of claim 13, wherein said organ is a heart, liver, kidney, or pancreas.

15. A method for identifying a candidate compound for reducing or preventing ischemic reperfusion injury, said method comprising:
(a) contacting a cell expressing an IKK-β gene with a candidate compound; and
(b) measuring IKK-β gene expression or IKK-β protein activity in said cell, wherein a candidate compound that reduces said expression or said activity, relative to IKK-β expression or activity in a cell not contacted with said candidate compound, is a candidate compound useful for reducing or preventing ischemic reperfusion injury.

16. The method of claim 15, wherein said IKK-β gene is an IKK-β fusion gene.

17. The method of claim 15, wherein step (b) comprises measuring expression of IKK-β mRNA or protein.

18. The method of claim 15, wherein said IKK-β is human IKK-β.

19. A method for identifying a candidate compound for reducing or preventing ischemic reperfusion injury, said method comprising:
(a) contacting IKK-β protein with a candidate compound; and
(b) determining whether said candidate compound binds said IKK-β protein, wherein a candidate compound that binds said IKK-β protein is a candidate compound useful for reducing or preventing ischemic reperfusion injury.

20. The method of claim 19, wherein said method further comprises testing said candidate compound for an ability to reduce expression of an IKK-β gene or activity of an IKK-β protein in a cell.

21. The method of claim 19, wherein said cell is a mammalian cell.

22. The method of claim 21, wherein said mammal is a rodent.

23. The method of claim 19, wherein said IKK-β is human IKK-β.

24. A kit comprising:
(a) a vector expressing a nucleic acid encoding a dominant negative IKK-β protein; and
(b) instructions for delivery of said vector to an organ for reducing or preventing ischemic reperfusion injury.

25. The kit of claim 24, wherein said organ is a heart.

26. The kit of claim 24, wherein said organ is a liver, kidney, or pancreas.

27. The kit of claim 24, wherein said organ is a human organ.

28. A vector comprising a polynucleotide encoding a dominant negative IKK-β protein operably linked to an organ-specific promoter.

29. The vector of claim 28, wherein said organ-specific promoter is a heart-specific promoter.

30. The vector of claim 29, wherein said promoter is a myosin heavy chain promoter.

31. The vector of claim 29, wherein said promoter is the MLC_{2V} promoter.

32. The vector of claim 28, wherein said organ-specific promoter is a kidney-specific promoter.

33. The vector of claim 28, wherein said organ-specific promoter is a liver-specific promoter.
